(19) United States
(12) Patent Application Publication
(10) Pub. No.: US 2019/0127705 A1
(43) Pub. Date: May 2, 2019

(54) METHOD FOR CULTURING MULTIPLE OVARIAN FOLLICLES BY USING ANGIOTENSIN II RECEPTOR AGONIST

(71) Applicant: SEOUL NATIONAL UNIVERSITY R&DB FOUNDATION, Seoul (KR)

(72) Inventors: Seung Yup KU, Seoul (KR); Yoon Young KIM, Seoul (KR)

(73) Assignee: SEOUL NATIONAL UNIVERSITY R&DB FOUNDATION, Seoul (KR)

(21) Appl. No.: 16/092,993

(22) PCT Filed: Apr. 14, 2017
(86) PCT No.: PCT/KR2017/004038

§ 371 Date: Oct. 11, 2018

(30) Foreign Application Priority Data
Apr. 14, 2016 (KR) 10-2016-0045642

(51) Int. Cl. C12N 5/071

(52) U.S. Cl. CPC C12N 5/0682; C12N 2501/998; C12N 2501/32

(57) ABSTRACT

A method for culturing multiple ovarian follicles by using an angiotensin II receptor agonist, including a first culturing step of culturing a cluster of separated multiple follicles in medium, and a second culturing step of further culturing the cluster of multiple follicles cultured in the culturing step, in medium containing an angiotensin II receptor agonist, and to a composition for culturing a cluster of multiple follicles. The composition contains an angiotensin II receptor agonist.

### CROSS REFERENCE TO PRIOR APPLICATIONS

This application is a National Stage Patent Application of PCT International Patent Application No. PCT/KR2017/004038 (filed on Apr. 14, 2017) under 35 U.S.C. § 371, which claims priority to Korean Patent Application No. KR 10-2016-0045642 (filed on Apr. 14, 2016), which are all hereby incorporated by reference in their entirety.

### BACKGROUND

The present disclosure relates to a method for culturing multiple ovarian follicles using an angiotensin II receptor agonist, and more specifically, to a method for culturing ovarian follicles including a first culturing step of culturing a cluster of separated multiple follicles in medium, and a second step of further culturing the cluster of multiple follicles cultured in the first culturing step in medium containing an angiotensin II receptor agonist, and to a composition for culturing a cluster of multiple follicles, wherein the composition contains an angiotensin II receptor agonist.

In modern society, infertility is increasing due to various factors such as environmental pollution and lifestyle changes, and, thus, it is also becoming a social issue. Various sterilization procedures and assisted reproductive technologies have been developed to solve this infertility problem. The most commonly used procedures are intrauterine insemination (IU, intrauterine sperm injection), in vitro fertilization-embryo transfer (IVF-ET, in vitro fertilization), and the like.

Among the methods of treating infertility, in case of the in vitro fertilization-embryo transfer, that is in case of the in vitro fertilization, an ovum obtained from natural ovulation or a superovulation injection and a sperm are fertilized in vitro and transplanted into a uterus. However, there is a problem about a side effect of the superovulation injection, and, thus, it is necessary to develop a method for obtaining an ovum via culturing a follicle in vitro.

The in vitro culturing of the follicle is an important biomechanical method for studying a factor that regulates ovum development in different stages and is also an alternative for conservation of the fertility. However, a method of culturing a follicle in vitro currently used by most researchers is a single-follicle culturing method, which is labor intensive and inefficient. Because in which only one follicle is sampled and transplanted in a test tube. Further, since an ovary tissue is denatured rapidly after removal, obtaining one follicle only from a cluster of follicles cut with a needle while viewing a microscope requires a high degree of skill.

In addition, during a follicle growth for ovulation in a body, usually one dominant follicle is selected and ovulated. In general, the dominant follicle discharges local secretion which inhibits growth of follicles around the dominant follicle (Reprod Fertil Dev., 23(3):444-67). Based on this phenomenon, only the in vitro culturing of the single-follicle is possible until now, and in vitro culturing of multiple follicles is difficult now. When it is possible to overcome the follicle inhibition around this dominant follicle, the in vitro culturing of the multiple follicles will be possible too. This may be a promising method of the biotechnology, which may obtain a large amount of the ova compared with the culturing multiple follicles collectively.

In this connection, angiotensin II plays a role in early maturation, degeneration and ovulation of the follicle (Med Princ Pract., 22(5):475-9), and is known as a local secretory factor acting via an angiotensin receptor present in the ovary. However, no studies have been conducted to determine whether the angiotensin II may be used for the in vitro culturing using a cluster of multiple follicles.

### SUMMARY

The present inventors have made extensive efforts to develop a method for obtaining a large number of follicles in culturing follicles in vitro. As a result of the efforts, it was confirmed that angiotensin II receptor agonist acts specifically on a cluster of multiple follicles joined with each other and grows and matures all follicles simultaneously. Further, it was confirmed that even when a matured ovum thus obtained from the follicle is fertilized with a sperm, a fertilization rate is similar to that of a mature follicle obtained by the existing single-follicle culturing, and cleavage of embryo is proceeded in a similar to that in the existing single-follicle culturing. Thus, the present disclosure was completed by confirming that a large number of ova may be obtained by culturing a cluster of multiple follicles in medium containing the angiotensin II receptor agonist.

A purpose of the present disclosure is to provide a method for culturing ovarian follicles including a first culturing step of culturing a cluster of separated multiple follicles in medium, and a second culturing step of further culturing the cluster of multiple follicles cultured in the culturing step, in medium containing an angiotensin II receptor agonist.

Another purpose of the present disclosure is to provide a composition for culturing cluster of multiple follicles, wherein the composition contains an angiotensin II receptor agonist.

Still another purpose of the present disclosure is to provide an angiotensin II receptor agonist for culturing a cluster of multiple follicles.

Using the method for culturing the cluster of multiple follicles according to the present disclosure may allow producing a large number of ova by culturing multiple follicles, such that the ova thus obtained may be usefully used for in vitro fertilization and infertility research, and the like.

### BRIEF DESCRIPTION OF THE DRAWINGS

FIG. 1 shows photographs that representing growth and maturation of follicles based on an angiotensin II receptor agonist treatment and an angiotensin II receptor antagonist treatment in culturing a single-follicle.

FIG. 2 is a graph showing ovulation rates of follicles based on an angiotensin II receptor agonist treatment and an angiotensin II receptor antagonist treatment in culturing a single-follicle.

FIG. 3 shows photographs that representing growth of follicles based on an angiotensin II receptor agonist treatment and an angiotensin II receptor antagonist treatment in culturing a cluster of multiple follicles.

FIG. 4 is a graph showing maturation rates of follicles based on an angiotensin II receptor agonist treatment and an angiotensin II receptor antagonist treatment in culturing a cluster of multiple follicles.

FIG. 5 is a graph comparing maturation rates of follicles based on an angiotensin II receptor agonist treatment in culturing a single-follicle and in culturing a cluster of multiple follicles.

FIG. 6 is a graph comparing fertilization rates of follicles based on angiotensin II receptor agonist treatment in culturing a single-follicle and in culturing a cluster of multiple follicles.

FIG. 7 shows photographs representing a progress of embryo cleavage in fertilization of a mature follicle obtained from culturing a single-follicle and culturing a cluster of multiple follicles. (a) of FIG. 7 shows a photograph representing a mature ovum obtained from culturing a single-follicle. (b) of FIG. 7 shows a photograph representing a mature ovum obtained from culturing a cluster of multiple follicles. (c) of FIG. 7 shows a photograph representing a fertilized embryo obtained from culturing a single-follicle. (d) of FIG. 7 shows a photograph representing a fertilized embryo obtained from culturing a cluster of multiple follicles.

FIG. 8 shows photographs representing growth and maturation of follicles obtained by culturing follicles collectively and culturing a cluster of multiple follicles.

### DETAILED DESCRIPTION

While developing a method for obtaining a large number of follicles in vitro culturing of the follicle, the present inventors found that an angiotensin II receptor agonist specifically acts on a cluster of multiple follicles including two to four follicles joined together, and simultaneously matures and grows all the follicles. In addition, it was confirmed that even when mature follicles from the multiple follicles are fertilized, a fertilization rate thereof was similar to that of a mature follicle from an existing single-follicle, and a cleavage of embryo proceeded similarly in the existing single-follicle. Therefore, it was found that a large number of follicles may be obtained by culturing the cluster of multiple follicles in medium containing the angiotensin II receptor agonist.

The present disclosure has significant implications in that when multiple follicles are cultured in a single-follicle form, growth and maturation of all follicles don't occur simultaneously even when those are treated with the angiotensin II receptor agonist, while, when the cluster of multiple follicles is cultured in the medium containing the angiotensin II receptor agonist, the growth and maturation of most follicles present in the cluster of multiple follicles are promoted.

This will be described in detail as follows. In this connection, each description and embodiment disclosed in the present disclosure may be applied to respective description and embodiment. That is, all combinations of the various elements disclosed in the present disclosure fall within the scope of the present disclosure. In addition, the specific disclosures set forth below do not limit the scope of the present disclosure.

In one embodiment for solving the above problem, the present disclosure provides a method for culturing ovarian follicles including a first culturing step of culturing a cluster of separated multiple follicles in medium, and a second further culturing step of culturing the cluster of multiple follicles cultured in the first culturing step, in medium containing an angiotensin II receptor agonist.

The term “follicle culturing,” as used in the present disclosure, refers to a process of raising a living follicle artificially in vitro under controlled conditions. Except for essential technical ideas provided in the present disclosure, the method for culturing the ovarian follicle may be performed without limitation using a method well known in the art.

A follicle culturing method of the present disclosure is particularly advantageous in that it may culture multiple follicles in vitro. Hereinafter, each step of the culturing method will be described in detail.

A first culturing step is a step of pre-culturing a cluster of separated multiple follicles in vitro. Via the first culturing step, a follicle present in vivo may be adapted to grow and mature in vitro.

The term “cluster of multiple follicles” used in the present disclosure means a cluster of multiple follicles joined together. A follicle grows in an ovary from an onset of menstruation until ovulation, during which the follicle grows passing through stages of a primordial follicle, a pre-antral follicle, an antral follicle, and a mature follicle. In the present disclosure, the cluster of multiple follicles may be in the pre-antral follicle stage, but is not limited thereto. The cluster of multiple follicles may be composed of two to four pre-antral follicles joined together, but the present disclosure is not limited thereto as long as four or more follicles are present in a joined state.

In the present disclosure, the follicle may be a mammalian follicle. Specifically, it may be, but is not limited to, a follicle of human, cow, sheep, pig, horse, rabbit, goat, mouse, hamster and rat.

In the present disclosure, the separation of the cluster of multiple follicles may be performed directly in a separated ovary or may be performed in the ovary in vivo, but is not limited thereto, and any method of follicle separation known in the art may be included in the scope of the present disclosure without limitation.

In one embodiment of the present disclosure, the separated ovary was placed in Hank's balanced salt solution (HBSS), then a 28-gauge syringe needle was used to separate the cluster of multiple follicles including two to four pre-antral follicles joined at beginning of development.

The term “medium” used in the present disclosure means medium capable of supporting growth and survival of cells including a follicle cell in vitro, and may include all conventional mediums used in the art suitable for a culture of the cells. A basic medium used for culture may include DMEM (Dulbecco's Modified Eagle's Medium), MEM (Minimal essential Medium), Opti-MEM, BME (Basal Medium Eagle), RPMI1640, F-10, F-12, αMEM (α Minimal essential Medium), GMEM (Glasgow's Minimal essential Medium) and Iscove's Modified Dulbecco's Medium, etc., but not limited thereto. The medium may further include, but is not limited to, insulin, bovine serum, recombinant follicle stimulating hormone, and recombinant luteinizing hormone, etc. in order to induce the growth and maturation of the follicle.

In addition, the medium of the present disclosure may further include a growth factor, hormone, polypeptide and an inorganic substance to aid in the culture and maintenance of the follicle cell. More specifically, the growth factor, the hormone, the polypeptide and the inorganic substance may be selected from a group consisting of bovine pituitary extract, an epidermal growth factor, insulin, hydrocortisone, transferrin, epinephrine, CaCl₂) and amphotericin B. The medium may also include an antibiotic such as penicillin, streptomycin or gentamicin to prevent bacterial infection.

In one specific embodiment of the present disclosure, after culture droplet was made on a culturing dish using culturing solution including the insulin, the bovine serum, the recombinant follicle stimulating hormone, the recombinant luteinizing hormone, a single pre-antral follicle or a cluster of multiple follicles collected from each droplet was seeded and cultured in an incubator at 37° C. in a 5% CO₂ for about 2 weeks.

Specifically, the first culture step may be performed for 2 days to 6 days, but is not limited thereto.

The second culturing step is a step of growing and maturing the follicle by culturing the cluster of multiple follicles cultured in the first culturing step in the medium containing the angiotensin II receptor agonist.

The term “angiotensin II” used in the present disclosure is a major regulator of blood pressure enhancement and aldosterone secretion, and is a hormone known to play an important role in regulating blood pressure and blood volume in cardiovascular disease. The angiotensin II may act through two receptors called angiotensin II receptor type 1 and angiotensin II receptor type 2.

The term “agonist” as used in the present disclosure refers to any compound that activates a biological response, for example activates a signaling pathway, by binding to a receptor to activate the receptor. Thus, the angiotensin II receptor agonist may refer to any compound that is bound to the angiotensin II receptor and activates the signaling pathway to be induced to the angiotensin II.

In the present disclosure, the angiotensin II receptor agonist may be one selected from a group consisting of for example angiotensin II, CGP-42112 and Novokinin trifluoroacetate salt, but it is not particularly limited thereto as long as it activates the signaling pathway to be induced to the angiotensin II by binding to the angiotensin II receptor.

In the follicle culturing method of the present disclosure, although there may be differences in a concentration and a culture period depending on which angiotensin II receptor agonist is selected, any angiotensin II receptor agonist capable of culturing the cluster of multiple follicles by applying it to the cluster is considered to be included in the scope of the present disclosure within the extent that the technical idea of the present disclosure is not changed.

In one example, a concentration of the CGP-42112 as the angiotensin II receptor agonist may range from 5 ug/mL to 20 ug/mL in medium. Specifically, the concentration may be from 5 ug/mL to 15 ug/mL in the medium, and more specifically, may be 10 ug/mL in the medium. However, as long as the cluster of multiple follicles is treated with CGP-42112 to effect on growth and maturation of all follicles in the cluster of multiple follicles, the concentration thereof is not limited to the above defined range.

In one specific embodiment of the present disclosure, the CGP-42112 was used as the angiotensin II receptor agonist, and the concentration thereof was adjusted to 10 ug/mL in the medium from a fifth day.

The term “angiotensin II receptor antagonist” used in the present disclosure refers to a drug that binds to a receptor to inhibit neurotransmitter release or hormone functions.

The term “growth” used in the present disclosure means that cells that make up the follicle multiply and an overall size of the follicle increases.

The term “maturation” used in the present disclosure means that a development of the follicle.

In addition, the follicle may be grown and maturated using the method for culturing the cluster of multiple follicles according to the present disclosure

The second culturing step may be performed for 5 days to 20 days, but is not limited thereto.

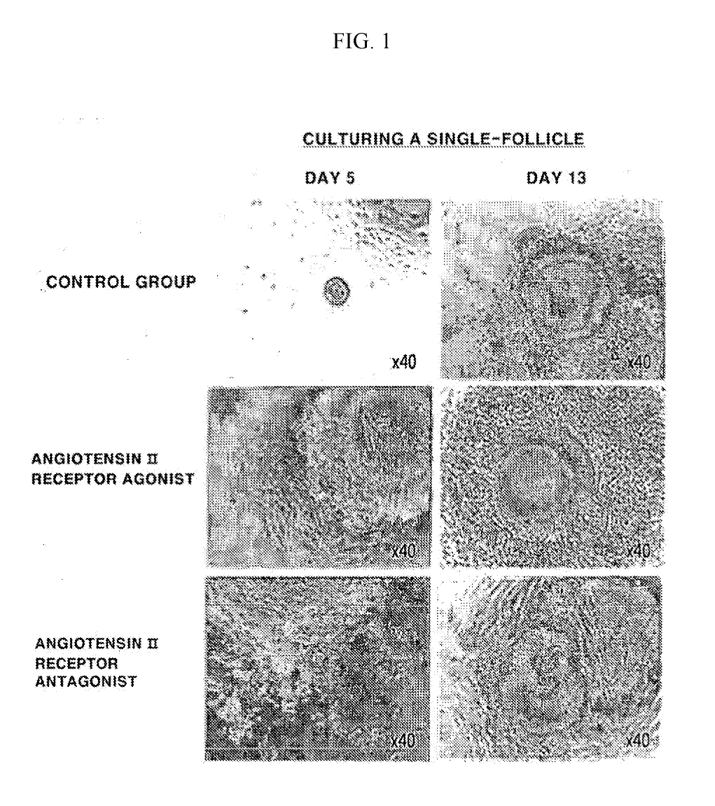

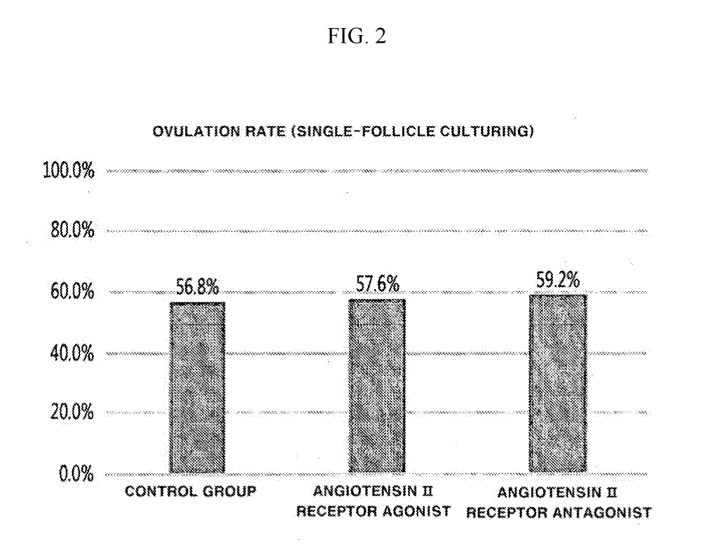

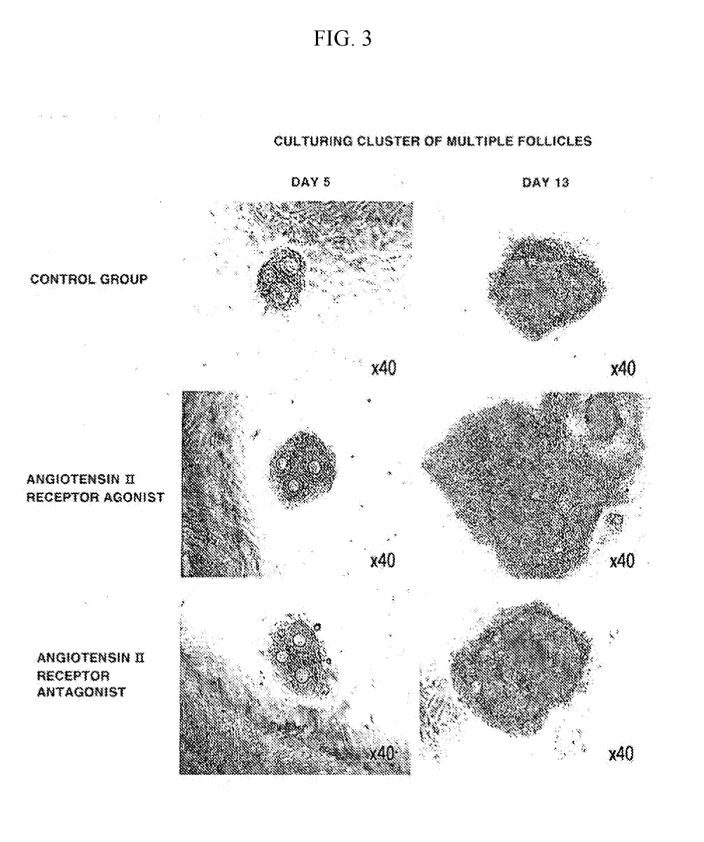

In one specific embodiment of the present disclosure, it was confirmed that, when no treatment is applied to the follicle, or when the follicles are single-follicle cultured by treating them with angiotensin II receptor agonist and angiotensin II antagonist, those has no effect on the growth and maturation of the single-follicles (see FIG. 1 to FIG. 2). In addition, it was confirmed that, when no treatment is applied to the cluster of multiple follicles, or when the cluster of multiple follicles is treated with angiotensin II receptor antagonist, several follicles in the cluster of multiple follicles were found to be unable to grow or to become apoptotic (see FIG. 3). On the other hand, it was confirmed that, when the cluster of multiple follicles is treated with angiotensin II receptor agonist, all follicles of the cluster were simultaneously promoted to grow (see FIG. 3).

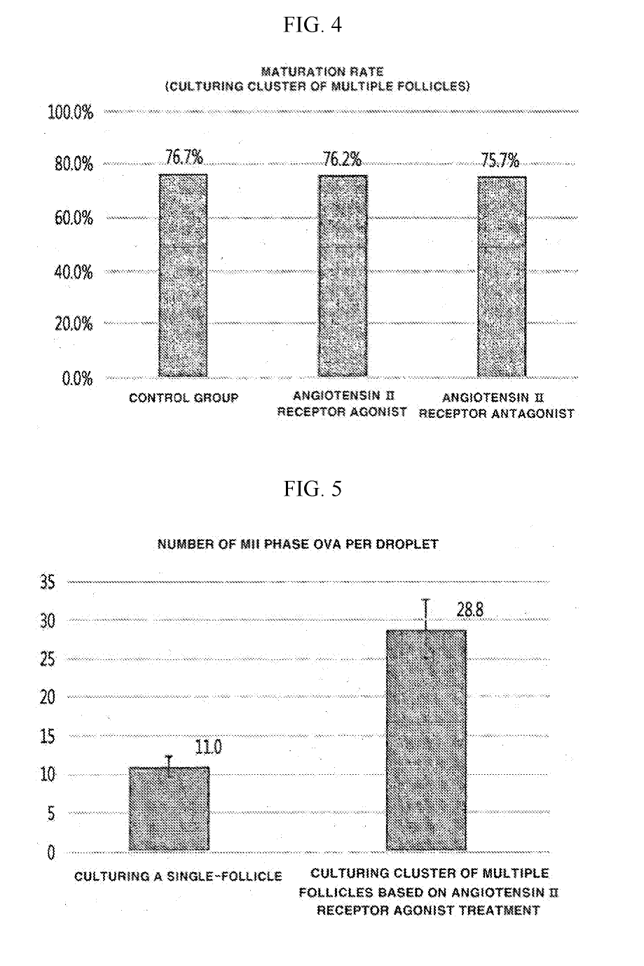

Meanwhile, it was confirmed that when no treatment is applied to the cluster of multiple follicles, or when the cluster of multiple follicles is treated with angiotensin II receptor antagonist or angiotensin II receptor agonist, maturation rates of the living follicles were similar (see FIG. 4). Thus, since the angiotensin II receptor agonist plays a role in promoting the growth of the follicles in culturing the cluster of multiple follicles, it was found that multiple follicles may be cultured simultaneously by treating them with the angiotensin II receptor agonist.

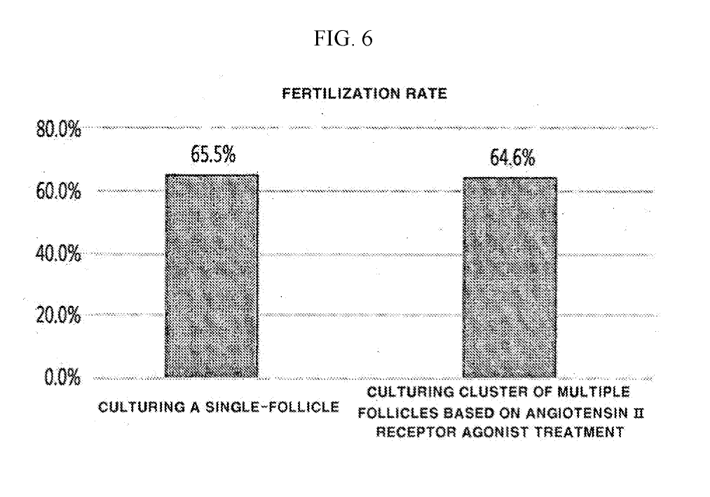

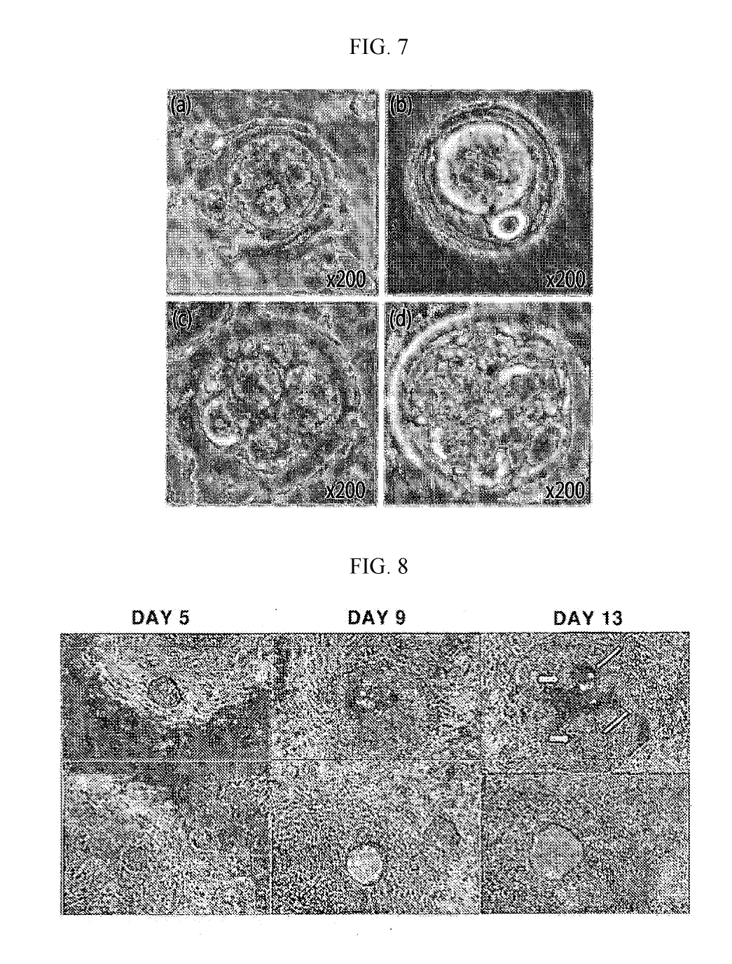

In another specific embodiment of the present disclosure, when the cluster of multiple follicles was treated with the angiotensin II receptor agonist, growth and maturation of the all follicles were promoted to a level similar to that in the single-follicle culturing under the same conditions (see FIG. 5). In addition, in case of fertilization with sperm using a mature follicle obtained from the follicle of the cluster of multiple follicles, cleavage of embryo was proceeded to a similar level compared with a case of fertilization with sperm using a single-follicle cultured mature follicle (see FIG. 6 and FIG. 7). Therefore, using the present disclosure method, since a large number of ova may be produced through the culturing of the multiple follicles, it is expected that ovum for in vitro fertilization may be efficiently produced in vitro and useful for various studied related to infertility.

In still another specific embodiment of the present disclosure, it was confirmed that when multiple follicles were cultured in a form of a single-follicle, treating them with angiotensin II receptor agonist did not result in simultaneous growth and maturation of all follicles, while when the multiple follicles were cultured via the method for culturing the cluster of the multiple-follicles of the present disclosure, growth and maturation of most follicles were promoted (see FIG. 8). Therefore, it was confirmed that the angiotensin II receptor agonist is able to grow and mature all follicles by specifically acting in culturing the cluster of multiple follicles.

The follicle culturing method of the present disclosure may further include obtaining an ovum by inducing ovulation in the cluster of multiple follicles cultured in the second culturing step.

The ovulation induction is a process of inducing the ovum to be released from a mature follicle. A substance used for the ovulation induction may include, but are not limited to, recombinant human chorionic growth hormone (rhCG), human epidermal growth factor (hEGF), human menopausal gonadotropin (hMG), and follicle stimulating hormone (FSH).

In one specific embodiment of the present disclosure, the cluster of multiple follicles was treated with the recombinant human chorionic growth hormone and the human epidermal growth factor to induce the ovulation.

In another embodiment, the present disclosure provides a composition containing angiotensin II receptor agonist for cluster of multiple follicles culturing.

In still another embodiment, the present disclosure provides for cluster of multiple follicles culturing applications of angiotensin II receptor agonist.

The angiotensin II, angiotensin II receptor agonist and cluster of multiple follicles are as described above.

In the present disclosure, even when the cluster of multiple follicles is treated with the angiotensin II receptor agonist to mature and grow all follicles simultaneously, and mature follicles from the follicles are fertilized with sperm, similar fertilization rate and similar proceeding of embryo cleavage were observed compared with fertilization using a mature follicle from a conventional single-follicle. Thus, the angiotensin II receptor agonist may be useful as the composition for the cluster of multiple follicles.

Hereinafter, the present disclosure will be described in more detail with reference to the following examples. However, these examples are provided to illustrate the present disclosure, and the scope of the present disclosure is not limited to these examples.

### Example 1. Separation of Pre-Antral Follicles from a Mouse

In order to separate pre-antral follicles to be used in an experiment, female C57BL6 mouse was used. In this connection, the mouse was given a light and dark period for 12 hours, and was allowed to freely drink water and eat food.

Specifically, an ovary was separated from a 14-day old mouse and placed in HBSS (Hank's balanced salt solution) containing 10% bovine serum. Then, pre-antral follicles in an early developmental stage were mechanically separated using a 28-gauge syringe needle. At this time, degenerated pre-antral follicles were removed, and healthy pre-antral follicles were used for a pre-antral follicle culturing. One separated pre-antral follicle was used for a single-follicle culturing, and a cluster including two to four pre-antral follicles joined together was used for culturing a cluster of multiple follicles.

### Example 2. In Vitro Culturing of the Separated Pre-Antral Follicles

In order to culture the separated pre-antral follicles in vitro, the following method was performed.

After 25 20 μl culture liquid droplets were made on each culture dish (Falcon 3000, BD Biosciences, 60×15 mm), the collected single pre-antral follicle, or the cluster of multiple follicles consisting of 2 to 4 joined pre-antral follicles were seeded to each droplet. Then, those follicles were cultured in a 5% CO₂ incubator at 37° C. for about 2 weeks. The culture liquid used here is Opti-MEM medium (Invitrogen) including insulin, 5% bovine serum, recombinant follicle stimulating hormone (FSH, Gonal-F®, Merck Serono, 0.2 IU/mL) and recombinant luteinizing hormone (LH, Luveris®, Merck Serono, 0.1 IU/mL). The culture liquid was changed in half with new medium every two days. In culturing process, a follicle morphology was observed using an inverted microscope (TE-2000; Nikon).

### Example 3. Regulation of an Angiotensin II Receptor

In order to observe a follicle culturing effect based on a regulation of the angiotensin II receptor, the single pre-antral follicle and the cluster of multiple follicles were compared and analyzed by divided into 3 groups: control group, angiotensin II receptor agonist (CGP-42112, Sigma)-treated group, and angiotensin II receptor antagonist (Saralasin, Sigma)-treated group. Specifically, the single pre-antral follicle and the cluster of multiple follicles were treated with the angiotensin II receptor agonist or the angiotensin II receptor antagonist to have a concentration of 10 ug/mL from day 5 of culturing.

### Example 4. Induction of Ovulation

On the 12th day of the culturing, it was observed that the pre-antral follicle was matured into an antral follicle. Then, the antral follicle was treated with recombinant human chorionic growth hormone, rhCG, Ovidrel®, Merck Serono) of 1.5 IU/m and human epidermal growth factor (hEGF) of 5 ng/mL in order to induce ovulation. At 16 to 18 hours after the ovulation induction, ovulation rate and maturation rate were calculated.

Specifically, the ovulation rate was calculated by counting the number of follicles ovulated after administration of the human chorionic growth hormone and the human epidermal growth factor among the seeded follicles, and the maturation rate was calculated by counting the number of ova in MII phase among the ovulated ova.

### Example 5. In Vitro Fertilization and Embryo Observation

In vitro fertilization was performed to evaluate fertilization rates of the ova obtained by the induction of ovulation. Specifically, a mature follicle ovulated in HTF (human tubal fluid) medium in the 5% CO₂ incubator at 37° C. was fertilized with a sperm collected from a 8 week old male C57BL6 mouse. When cell division occur or two pronuclei are seen and a second polar body is observed, the ovum is regarded as fertilized.

### Experimental Example 1. Investigation of Effect of Angiotensin II in Culturing a Single-Follicle

In order to investigate effect of angiotensin II in culturing a single-follicle, growth and maturation rates based on the treatment with angiotensin II receptor agonist or angiotensin II receptor antagonist were intended to be investigated. To this end, the growth of the follicles was observed in culturing of the single-follicle by treating with angiotensin II receptor agonist or angiotensin II receptor antagonist via the method of Example 1 to Example 4. Further, ovulation rates were investigated by inducing ovulation in the cultured follicles.

As a result of the experiment, when comparing the growth of the follicle on the fifth day and the growth of the follicle on the 13th day, a single-follicle treated with angiotensin II receptor agonist (AT II receptor agonist) or angiotensin II receptor antagonist (AT II receptor antagonist) was observed to be growing at a level similar to a single-follicle (control group, CTL) that had not been treated (see FIG. 1). In addition, the ovulation rate of a single-follicle treated with angiotensin II receptor agonist or angiotensin II receptor antagonist was not significantly different from that of a single-follicle that had not been treated (see FIG. 2).

In conclusion, it was confirmed that the angiotensin II did not affect growth and maturation of the follicle in the culturing a single-follicle.

### Experimental Example 2. Investigation of Effect of Angiotensin II in Culturing a Cluster of Multiple Follicles

Next, effect of angiotensin II on cluster of multiple follicles including two to four follicles joined together in culturing the cluster of multiple follicles was intended to be investigated. Growth of follicles was observed during the culturing of the cluster of the follicles by treating with angiotensin II receptor agonist or angiotensin II receptor antagonist via the method of Example 1 to Example 4. Further, maturation rates based on living follicles of each cluster of multiple follicles were investigated.

As a result of the experiment, it was observed that when comparing the growth of the cluster of multiple follicles on the fifth day and the growth of the cluster of multiple follicles on the 13th day, in a control group that had not been treated, some of the follicles of the cluster of multiple follicles did not undergo growth and almost little follicle fluid was accumulated in the follicle. However, it was observed that, in the cluster of multiple follicles treated with the angiotensin II receptor agonist, the growth of all follicles was significantly promoted and the accumulation of the follicle fluid in the follicle was increased. On the other hand, it was found that some follicles in the cluster of multiple follicles treated with the angiotensin II receptor antagonist did not grow simultaneously but fell into apoptosis during development (see FIG. 3).

Meanwhile, as a result of checking maturation rates of live follicles in culturing the cluster of multiple follicles, the cluster of multiple follicles treated with angiotensin II receptor agonist (AT II receptor agonist) or angiotensin II receptor antagonist (AT II receptor antagonist) showed maturation rates similar to that of a single-follicle (control group, CTL) that had not been treated (see FIG. 4). Therefore, it was confirmed that the maturation rate of the follicle and the angiotensin II were not correlated with each other.

In conclusion, it was confirmed that some the cluster of multiple follicles that had not been treated and some follicles of the cluster of multiple follicles treated with the angiotensin II receptor antagonist didn't grow, but all follicles of the cluster of multiple follicles treated with the angiotensin II receptor agonist were promoted to grow, such that it is possible to culture multiple follicles simultaneously by treating them with the angiotensin II receptor agonist.

### Experimental Example 3. Comparison of Maturation Rates and Fertilization Rates of Follicles in Culturing Multiple Follicles Collectively and in Culturing Cluster of Multiple Follicles

In order to further confirm the effect of the angiotensin II in culturing the cluster of multiple follicles, maturation rate and fertilization rate were intended to be checked. Mature follicles were ovulated by treating them with the angiotensin II receptor agonist and inducing the ovulation via the method of Example 1 to Example 5. Then, the ovulated mature follicles were fertilized with sperms.

As a result of the experiment, mature follicles were obtained by culturing multiple follicles simultaneously during the culturing of the cluster of multiple follicles. A maturation rate (26.8%) was found to be significant with a maturation rate (11%) of the single-follicle (see FIG. 5). In addition, as a result of fertilizing the matured follicles, fertilization rates of the single-follicle culturing and culturing of the cluster of multiple follicles showed no significant difference (see FIG. 6), and a progression of cleavage of an embryo was observed (see FIG. 7).

In conclusion, it was confirmed that when the cluster of multiple follicles is cultured by treating them with the angiotensin II receptor agonist, all follicles grow and mature up to a similar level to that of a follicle cultured via a single follicle culturing under the same condition. In addition, when the ovum obtained from the follicle via the cluster culturing was fertilized with the sperm, a level of fertilization thereof is achieved at the same level of that from the ovum from the single follicle culturing. Therefore, a method for culturing the cluster of multiple follicles in accordance with the present disclosure allows culturing of multiple follicles. Thus, ovum may be produced in large quantities, and ovum necessary for in vitro fertilization may be efficiently produced in vitro. Thus, this method may be expected to be useful for various studies related to infertility.

### Experimental Example 4. Comparison of Maturation Rates of Follicles in Culturing Multiple Follicles Collectively and in Culturing the Cluster of the Multiple Follicles

In order to further clarify the effect of the method for culturing the cluster of multiple follicles established in the present disclosure, the method for culturing the cluster of the multiple-follicles was intended to be compared with the case of culturing multiple follicles collectively. To this end, the growth of the follicles was observed during collective-culturing of the multiple follicles or during culturing the cluster of multiple follicles via treating them with angiotensin II receptor agonist in accordance with the method of Example 1 to Example 4. Further, maturation rates were investigated by inducing ovulation in the cultured follicles.

As a result of the experiment, it was confirmed that when the multiple follicles are collectively treated with the angiotensin II receptor agonist, all follicles promoted to grow but fell into apoptosis during development. Further, it was found that there was almost no follicle fluid accumulation in the follicles (see bottom of FIG. 8).

On the other hand, it was confirmed that, when the cluster of multiple follicles was treated with the angiotensin II receptor agonist, all follicles present in the cluster of multiple follicles were significantly promoted to grow (see top of FIG. 8). In addition, follicle fluid accumulation in the follicle (white arrow) and healthy shape of mature follicle (yellow arrow) were clearly observed in the all follicles present in the cluster of multiple follicles (see top of FIG. 8).

Therefore, the above results confirm that when the multiple follicles were cultured collectively by treating them with the angiotensin II receptor agonist, the growth and maturation of all follicles don't occur simultaneously, and furthermore, culturing the multiple follicles collectively may be not advantageous. On the other hand, it was found that since the method for culturing the cluster of the multiple-follicles in accordance with the present disclosure promotes the growth and maturation of most follicles, the angiotensin II receptor agonist is capable of growing and maturing all follicles healthily by specifically acting on the cluster of multiple follicles culturing.

In this specification, the description of the present disclosure may omit certain information known to those skilled in the art. In addition to specific embodiments disclosed in the present disclosure, various modifications may be made without departing from the technical idea or essential composition of the present disclosure. Accordingly, the present disclosure may be embodied in a manner and forms other than those specifically described and illustrated herein, which is understandable to those of ordinary skill in the art of the present disclosure.

1. A follicle culturing method comprising:
(a) a first culturing step of culturing a cluster of separated multiple follicles in medium; and
(b) a second culturing step of further culturing the cluster of multiple follicles cultured in the first culturing step, in medium containing an angiotensin II receptor agonist.

2. The follicle culturing method of claim 1, wherein the cluster of separated multiple follicles is in a pre-antral stage.

3. The follicle culturing method of claim 1, wherein the cluster of multiple follicles includes two to four pre-antral follicles joined together.

4. The follicle culturing method of claim 1, wherein the angiotensin II receptor agonist is one selected from a group consisting of angiotensin II, CGP-42112 and Novokinin trifluoroacetate salt.

5. The follicle culturing method of claim 4, wherein a concentration of the CGP-42112 is in a range of 5 ug/mL to 20 ug/mL in the medium.

6. The follicle culturing method of claim 1, wherein the first culturing step is performed for 2 days to 6 days.

7. The follicle culturing method of claim 1, wherein the second culturing step is performed for 5 days to 20 days.

8. The follicle culturing method of claim 1, further including a step of obtaining an ovum by inducing ovulation of the cluster of multiple follicles cultured in the second culturing step.

9. A composition for culturing a cluster of multiple follicles, wherein the composition contains an angiotensin II receptor agonist.

10. The composition of claim 9, wherein the cluster of multiple follicles includes two to four pre-antral follicles joined together.

11. The composition of claim 9, wherein the angiotensin II receptor agonist is one selected from a group consisting of angiotensin II, CGP-42112 and Novokinin trifluoroacetate salt.

12. The composition of claim 9, wherein a concentration of the CGP-42112 is in a range of 5 ug/mL to 20 ug/mL in medium.
